(19) United States
(12) Patent Application Publication
(10) Pub. No.: US 2015/0057505 A1
(43) Pub. Date: Feb. 26, 2015

(54) Method for Administering a Medicament to a Fetus

(71) Applicant: Elwha LLC, Bellevue, WA (US)

(72) Inventors: Mahalaxmi Gita Bangera, Renton, WA (US); Roderick A. Hyde, Redmond, WA (US)

(21) Appl. No.: 13/973,706

(22) Filed: Aug. 22, 2013

(51) Int. Cl. A61M 5/172; A61M 31/00; A61B 5/145; A61B 5/05; A61B 5/01; A61M 37/00; A61B 5/00

(52) U.S. Cl. CPC A61M 5/1723; A61M 37/0015; A61M 31/002; A61B 5/4362; A61B 5/05; A61B 5/01; A61B 5/14503
USPC 600/301; 604/506; 604/501; 604/22; 604/503; 600/300; 600/573; 600/407; 600/549; 600/309

(57) ABSTRACT

Methods are disclosed herein for delivering a medicament to a fetus. The method includes administering the medicament from an injector configured to deliver the medicament across an amniotic membrane to amniotic fluid of the fetus. The method includes administering the medicament from a transmembrane patch placed at an amniotic membrane configured to deliver the medicament across the amniotic membrane to amniotic fluid of the fetus.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application is related to and/or claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)). In addition, the present application is related to the “Related Applications,” if any, listed below.

### PRIORITY APPLICATIONS

None.

### RELATED APPLICATIONS

None.

The United States Patent Office (USPTO) has published a notice to the effect that the USPTO's computer programs require that patent applicants reference both a serial number and indicate whether an application is a continuation, continuation-in-part, or divisional of a parent application. Stephen G. Kunin, Benefit of Prior-Filed Application, USPTO Official Gazette Mar. 18, 2003. The USPTO further has provided forms for the Application Data Sheet which allow automatic loading of bibliographic data but which require identification of each application as a continuation, continuation-in-part, or divisional of a parent application. The present Applicant Entity (hereinafter “Applicant”) has provided above a specific reference to the application(s)from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant has provided designation(s) of a relationship between the present application and its parent application(s) as set forth above and in any ADS filed in this application, but expressly points out that such designation(s) are not to be construed in any way as any type of commentary and/or admission as to whether or not the present application contains any new matter in addition to the matter of its parent application(s).

If the listings of applications provided above are inconsistent with the listings provided via an ADS, it is the intent of the Applicant to claim priority to each application that appears in the Priority Applications section of the ADS and to each application that appears in the Priority Applications section of this application.

All subject matter of the Priority Applications and the Related Applications and of any and all parent, grandparent, great-grandparent, etc. applications of the Priority Applications and the Related Applications, including any priority claims, is incorporated herein by reference to the extent such subject matter is not inconsistent herewith.

### SUMMARY

Methods are disclosed herein for delivering a medicament to a fetus. An approach to a more effective medical treatment of a fetus involves a delivering a therapeutic composition directly to the fetus and bypassing administration of the therapeutic composition directly to the mother. The method avoids maternal administration of the therapeutic composition that normally relies upon transplacental transfer through the mother to the fetus. With the introduction of high-resolution ultrasonography, a method for administering a therapeutic composition to a fetus may include bypassing the placenta through direct administration across the amniotic membrane to the amniotic fluid. Direct treatment across the amniotic membrane may avoid maternal toxicity and the metabolic effects of administered agents.

A method for delivering a medicament to a fetus is disclosed that includes administering the medicament across an amniotic membrane to amniotic fluid of the fetus from an injector non-invasively positioned to the amniotic fluid.

The method includes administering the medicament from the injector surgically emplaced at an outer wall of the amniotic membrane. The method includes administering the medicament by one or more of microneedle, microjet, microcapsules, iontophoresis, and sonophoresis. The method includes laproscopically placing the injector at the outer wall of the amniotic membrane. The injector may be configured to be placed laproscopically at the outer wall of the amniotic membrane. The injector may be configured to be placed for one-time delivery. The injector may include a transmembrane patch. The method transmembrane patch may be configured to deliver the medicament by one or more of microneedle, microjet, microcapsules, iontophoresis, and sonophoresis. The transmembrane patch may be configured to deliver the medicament in response to a delivery schedule. The transmembrane patch may be configured to deliver the medicament in response to an external command.

In some embodiments, the method includes placing a sensor configured to sense one or more physiological conditions in proximity to the fetus. The method includes initiating a signal to control administration of the medicament regulated by a controller in response to the one or more sensed physiological conditions. The method includes administering the medicament at maternal epithelium by one or more of microjets and microneedles. The method includes administering the medicament at maternal epithelium by microcapsules. The method includes administering the medicament at maternal epithelium by one or more of iontophoresis and sonophoresis. The medicament may be formulated for the fetal gastrointestinal tract. The medicament may be formulated for intramembranous fetal transfer. The medicament may be formulated to be embedded in microcapsules. The medicament may be formulated for extended release characteristics.

A method for delivering a medicament to a fetus is disclosed that includes administering the medicament from a transmembrane patch placed at an amniotic membrane configured to deliver the medicament across the amniotic membrane to amniotic fluid of the fetus. The method includes surgically emplacing the transmembrane patch at the amniotic membrane. The method includes surgically resealing tissue over the transmembrane patch. The method includes laproscopically emplacing the transmembrane patch at the amniotic membrane. The transmembrane patch may be placed for a one-time event for each delivery.

In some embodiments, the method includes placing a sensor in proximity to the fetus to detect one or more physiological conditions of the fetus. The sensor may be incorporated with the transmembrane patch. The method includes initiating a signal to detect with the sensor an analyte in an amniotic fluid sample. The method includes initiating a signal to detect the one or more physiological conditions of the fetus by vibrational sensing. The method includes initiating a signal to detect the one or more physiological conditions of the fetus by electrical sensing. The method includes initiating a signal to detect the one or more physiological conditions of the fetus by electromagnetic sensing. The method includes initiating a signal to control administration of the medicament regulated by a controller in response to the one or more sensed physiological condition. The method includes administering the medicament by one or more of microneedle injection, microjet, microcapsules, iontophoresis, and sonophoresis. The transmembrane patch may be configured to deliver the medicament in response to a delivery schedule. The transmembrane patch may be configured to deliver the medicament in response to an external command.

A method for delivering a medicament to a fetus is disclosed that includes administering the medicament across an amniotic membrane to amniotic fluid of the fetus from one or more of microjet injectors and microneedle injectors non-invasively positioned to the amniotic fluid. The one or more of microjet injectors and microneedle injectors may be laproscopically placed at an outer wall of the amniotic membrane.

A method for delivering a medicament to a fetus is disclosed that includes administering the medicament from microcapsules injected into amniotic fluid of the fetus. The method includes initiating a signal to a controller to inject the microcapsules through an amniotic membrane by a needleless injector utilizing at least one of a microjet, sonophoresis, or iontophoresis. The method includes laproscopically placing the needleless injector at an outer wall of the amniotic membrane. The method includes initiating a signal to a controller to inject the microcapsules through the amniotic membrane by a needle-based injector non-invasively positioned to the amniotic fluid. The method includes initiating a signal to a controller to inject the microcapsules transdermally through the maternal skin and through the amniotic membrane by the needle-based injector. The method includes laproscopically placing the needle-based injector at an outer wall of the amniotic membrane. The method includes initiating a signal to a controller to transdermally inject the microcapsules through the amniotic membrane to the amniotic fluid by a needle-based injector. The microcapsules may be formulated for extended release of the medicaments.

A method for detecting one or more physiological conditions of a fetus is disclosed that includes placing a device including a transmembrane sensor in contact with an outer wall of an amniotic membrane of the fetus; and initiating a signal to the transmembrane sensor and a controller of the device to detect the one or more physiological conditions of the fetus. The method includes initiating a signal to the sensor and controller to detect the one or more physiological conditions by removing an analyte through the amniotic membrane. The method includes initiating a signal to the sensor and controller to remove the analyte through the amniotic membrane by microneedle, sonophoresis, or iontophoresis.

In some embodiments, the device may be configured to detect the one or more physiological conditions by sensing vibrations caused by the fetus or surrounding amniotic tissue. The device may be configured to detect the one or more physiological conditions by sensing electrical signals from the fetus or surrounding amniotic tissue. The device may be configured to detect the one or more physiological conditions by sensing electromagnetic signals from the fetus or surrounding amniotic tissue. The device may be configured to detect one or more of pH, temperature, analyte identity, or analyte concentration. The device may be configured to wirelessly report the sensed physiological condition to a remote computing device. The device may be configured to surgically reseal tissue over the transmembrane sensor in contact with the outer wall of the amniotic membrane. The device may be configured to report the sensed physiological condition to a computing device on a predetermined schedule. The device may be configured to report the sensed physiological condition to a computing device in response to one or more queries. The device may be configured to report the sensed physiological condition to a computing device based on previous measurements of the physiological condition. In some embodiments, the method includes initiating a signal to the controller to administer a medicament across the amniotic membrane from an injector non-invasively positioned to the amniotic fluid in response to measurements of one or more sensed physiological conditions.

A method for delivering a medicament to a fetus is disclosed that includes placing a device including a transmembrane sensor in contact with an outer wall of an amniotic membrane of the fetus; initiating a signal to the transmembrane sensor and a controller of the device to detect one or more physiological conditions of the fetus; and initiating a signal the device and the controller to administer the medicament across an amniotic membrane to amniotic fluid of the fetus from an injector non-invasively positioned to the amniotic fluid responsive to one or more sensed physiological conditions.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a schematic representation of a drug delivery device including a patch on an amniotic membrane to deliver one or more medicaments to a fetus.

FIG. 2 is a schematic representation of an amniotic membrane including epithelial layer, amnion layer, intermediate layer, and chorion layer and the extent of microneedle penetration through the amniotic membrane.

FIG. 3 is a schematic representation of a drug delivery device in a method for delivering a medicament to a fetus.

FIG. 4 illustrates an exemplary method for delivering a medicament to a fetus.

FIG. 5 illustrates an exemplary method for delivering a medicament to a fetus.

FIG. 6 illustrates an exemplary method for delivering a medicament to a fetus.

FIG. 7 illustrates an exemplary method for delivering a medicament to a fetus.

FIG. 8 illustrates an exemplary method for detecting one or more physiological conditions of a fetus.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Methods are disclosed herein for delivering a medicament to a fetus. An approach to a more effective medical treatment of a fetus involves a method that includes delivering a therapeutic composition directly to the fetus and bypassing administration of the therapeutic composition directly to the mother. The method avoids maternal administration of the therapeutic composition that normally relies upon transplacental transfer through the mother to the fetus. It has been shown, for example, that the passage of therapeutic cardiac treatment across the placenta is less efficient to treat hydropic failure in the fetus, thus rendering the sickest fetuses least likely to benefit. With the introduction of high-resolution ultrasonography, a method for administering a therapeutic composition to a fetus may include bypassing the placenta through direct administration across the amniotic membrane to the amniotic fluid. The advantages of direct treatment are the avoidance of maternal toxicity and the metabolic effects of administered agents.

A method for delivering a medicament to a fetus includes administering the medicament from an injector configured to deliver the medicament across an amniotic sac or amniotic membrane to amniotic fluid of the fetus. The amniotic sac, also referred to as the amniotic membrane, is the sac in which the fetus develops in amniotes. It is a tough but thin transparent pair of membranes, which hold a developing embryo (and later fetus) until shortly before birth. The inner membrane, the amnion, contains the amniotic fluid and the fetus. The outer membrane, the chorion, contains the amnion and is part of the placenta. Its wall is the amnion, the inner of the two fetal membranes. It encloses the amniotic cavity and the embryo. The amniotic cavity contains the amniotic fluid. On the outer side, the amniotic sac is connected to the yolk sac, to the allantois and, through the umbilical cord, to the placenta. The medicament may be formulated for intramembranous fetal transfer, e.g., through the amniotic membrane, maternal or fetal skin, placenta, or umbilical cord.

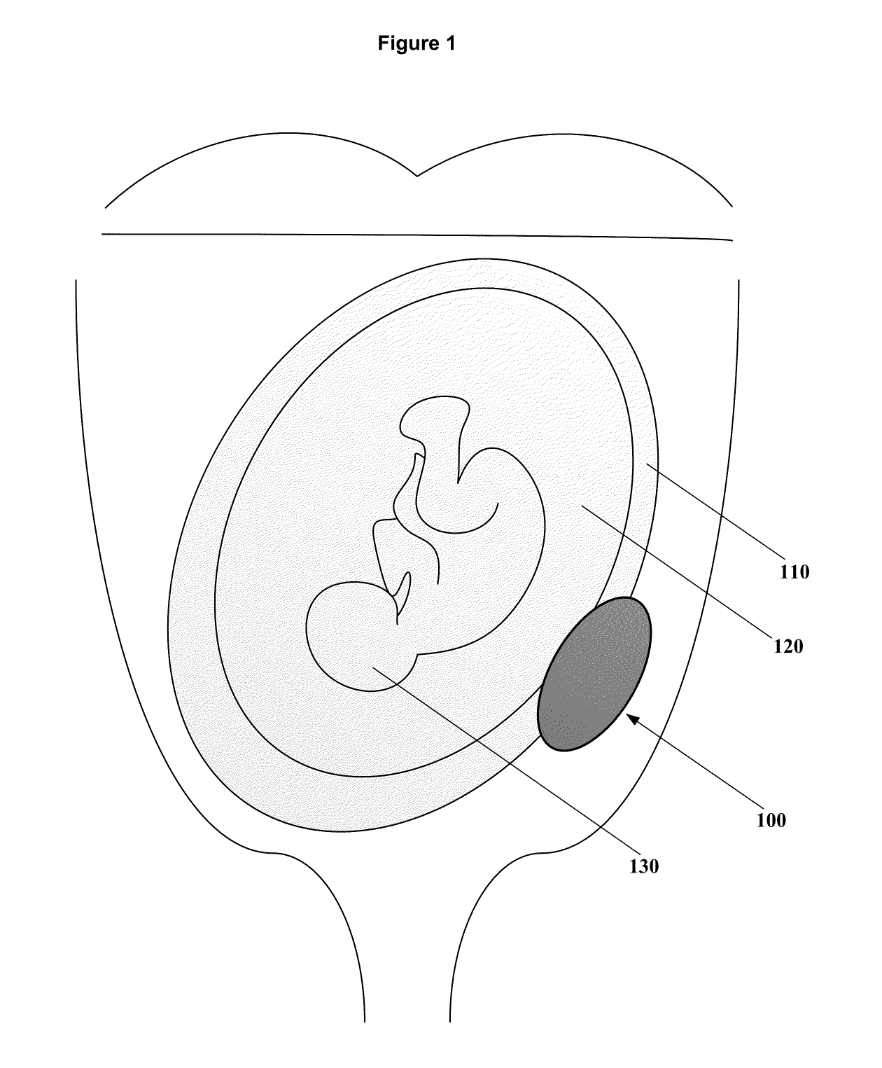

FIG. 1 depicts a diagrammatic view of an aspect of a drug delivery patch 100 in a method for delivering a medicament to a fetus 130. A drug delivery patch 100 containing the medicament is implanted on the external (outermost) surface 110 of the amniotic sac. The patch 100 may be surgically implanted on the amniotic sac 110 and deliver the medicament transmembrane across the amniotic sac to the amniotic fluid 120 for days or weeks for delivery of the medicament to the fetus 130. See e.g., Patel et al., “Transdermal Drug Delivery System: A Review,” The Pharma Innovation, Volume 1, No. 4, pages 66-75, 2012 and U.S. Pat. No. 6,726,920 issued to Theeuwes et al. on Apr. 27, 2004, which are incorporated herein by reference.

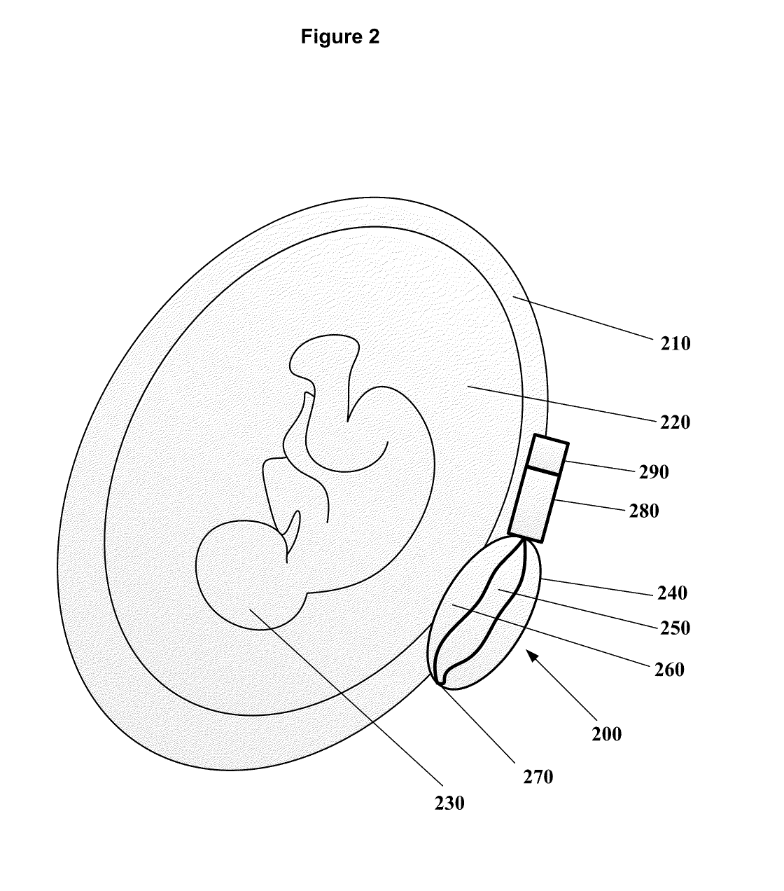

FIG. 2 depicts a diagrammatic view of an aspect of a drug delivery patch 200 in a method for delivering a medicament to a fetus 230. The patch 200 is constructed with a backing membrane 240, a reservoir 250 containing the medicament, a medicament-permeable membrane 260, and an adhesive 270 which adheres to the amniotic sac and releases the medicament across the amniotic sac membranes 210 into the amniotic fluid 220 over an extended period to deliver the medicament to the fetus 230. For example a drug delivery patch 200 may be fabricated with: (1) an outer layer or backing 240 of polyurethane which is impervious to the medicament and body fluids; (2) a membrane of polypropylene 260 which is permeated by the medicament and (3) an adhesive 270 (e.g., a thrombin-based sealant, Vitex, available from V.I. Technologies, NY). The drug delivery patch 200 may further include a sensor 280 to sense the presence or level of one or more physiological conditions or parameters, for example, fetal tachycardia, respiratory distress syndrome, thyroid disease, body temperature, respiration rate, pulse, blood pressure, edema, oxygen saturation, pathogen levels, inflammatory mediators, cytokines, or toxin levels. The drug delivery patch 200 may further include a sensor 280 and a controller 290 to detect the one or more physiological conditions and to control administration of the medicament across the amniotic membrane.

The drug delivery device includes a drug delivery patch 200 containing the medicament implanted on the external (outermost) surface of the amniotic sac 210. The drug delivery patch 200 releases the medicament across the amniotic sac membrane 210 into the amniotic fluid over an extended period. The medicament may be a pharmaceutical formulation of a drug or biologic. The medicament may be formulated in a microparticle or nanoparticle containing the drug or biologic. The pharmaceutical formulation of the drug or biologic may cross the amniotic sac membrane 210 by various treatments including microneedles, microparticles, iontophoresis, sonophoresis, or membrane permeable chemical components.

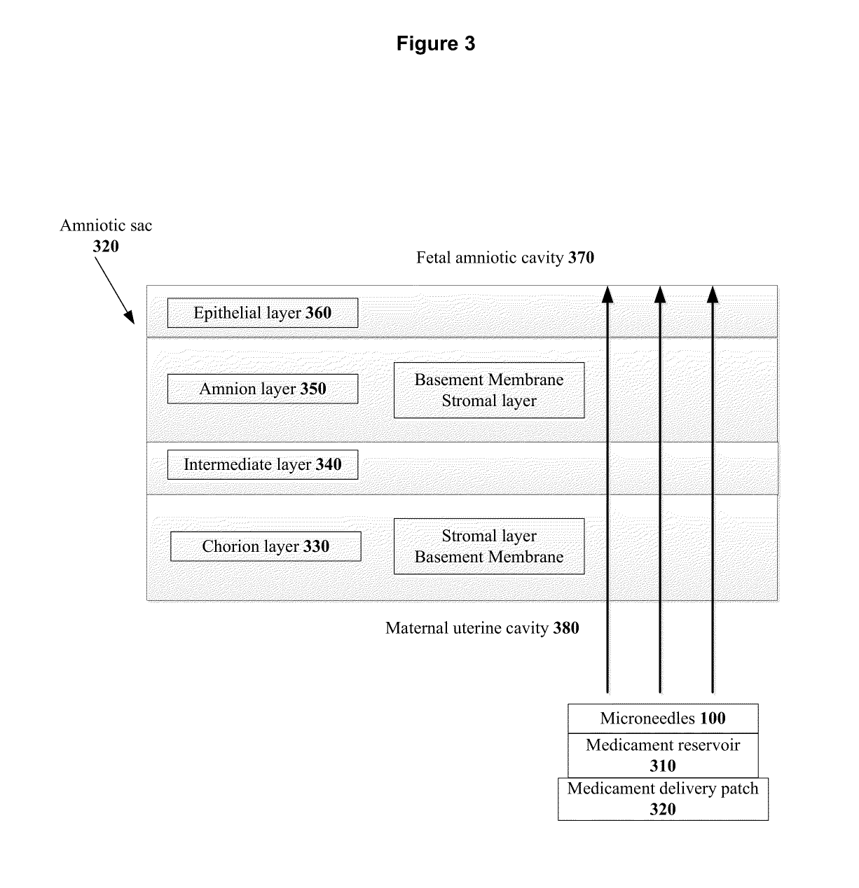

FIG. 3 depicts a diagrammatic view of an aspect of a method for delivering a medicament to a fetus that includes microneedles 300 attached to a drug delivery patch 320 and medicament reservoir 320 located in the maternal uterine cavity 380 on an amniotic sac 320. The microneedles 300 may penetrate the chorion layer 330, the intermediate layer 340 the amnion layer 350 and the epithelial layer 360 of an amniotic sac 320. The microneedles 300 are comprised of polymers and containing a medicament to be delivered to fetal amniotic cavity 370 containing the amniotic fluid and to the fetus. The microneedles 300 convert from a rigid needle structure to a hydrogel once inserted in the amniotic sac 320 and release medicament into the amniotic fluid 370. Polymeric, degradable microneedle arrays may be designed with preferred release kinetics. See e.g., U.S. Patent Application No. 2011/0195124, which is incorporated herein by reference.

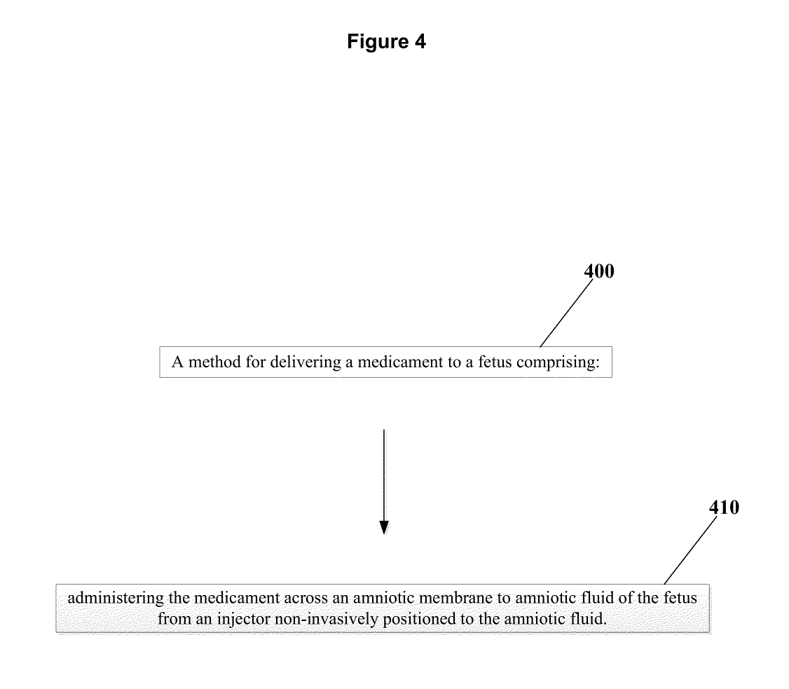

FIG. 4 illustrates an exemplary method 400 for delivering a medicament to a fetus including administering 410 the medicament from an injector configured to deliver the medicament across an amniotic membrane to amniotic fluid of the fetus.

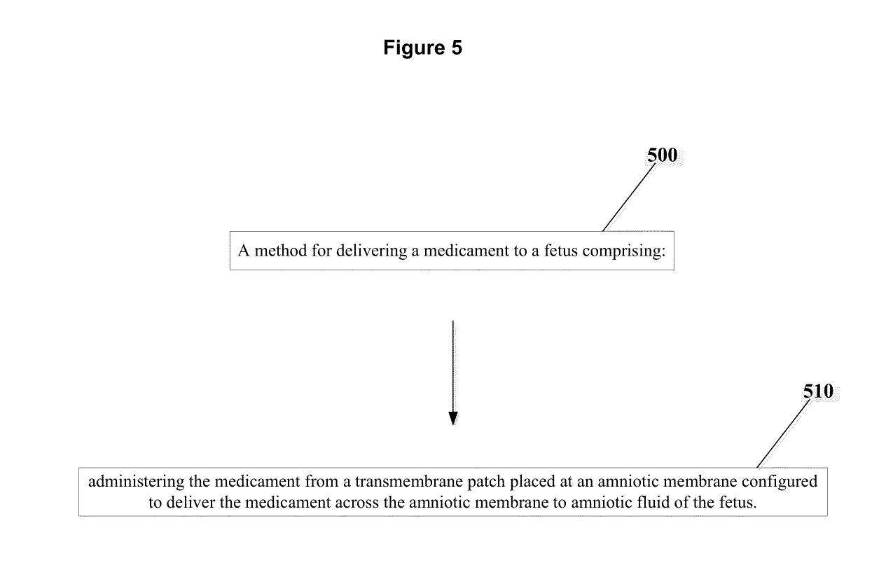

FIG. 5 illustrates an exemplary method 500 for delivering a medicament to a fetus including administering 510 the medicament from a transmembrane patch placed at an amniotic membrane configured to deliver the medicament across the amniotic membrane to amniotic fluid of the fetus.

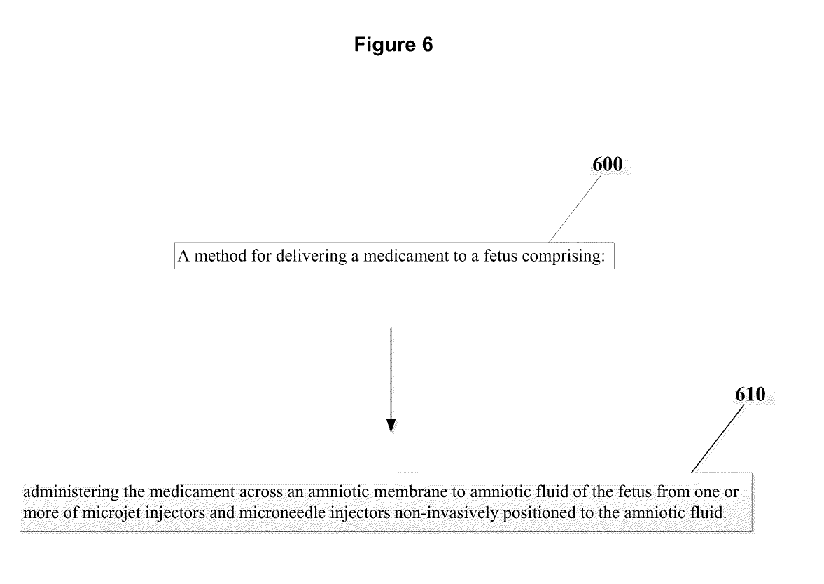

FIG. 6 illustrates an exemplary method 600 for delivering a medicament to a fetus including administering 610 the medicament from one or more of microjet injectors and microneedle injectors placed at an amniotic membrane configured to deliver the medicament across the amniotic membrane to amniotic fluid of the fetus.

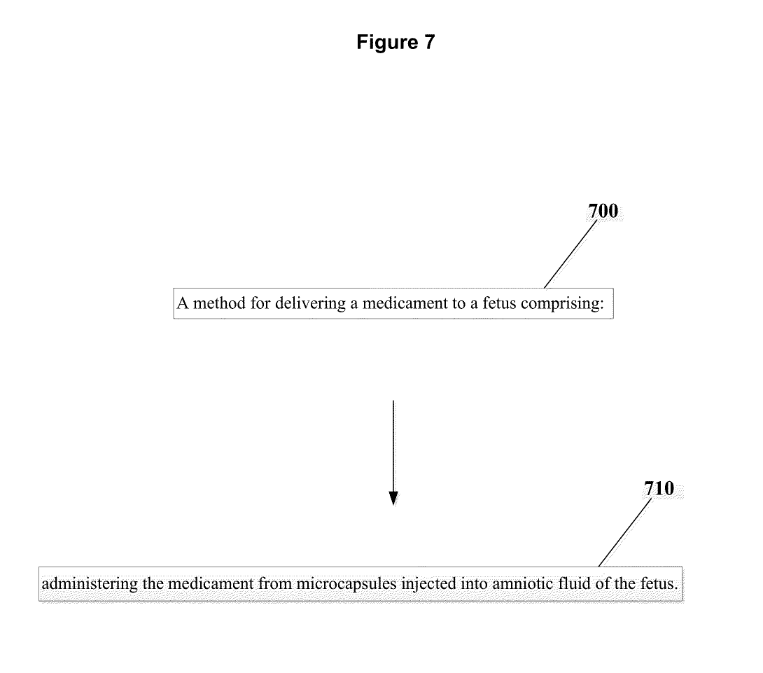

FIG. 7 illustrates an exemplary method 700 for delivering a medicament to a fetus including administering 710 the medicament from microcapsules placed at an amniotic membrane configured to deliver the medicament across the amniotic membrane to amniotic fluid of the fetus.

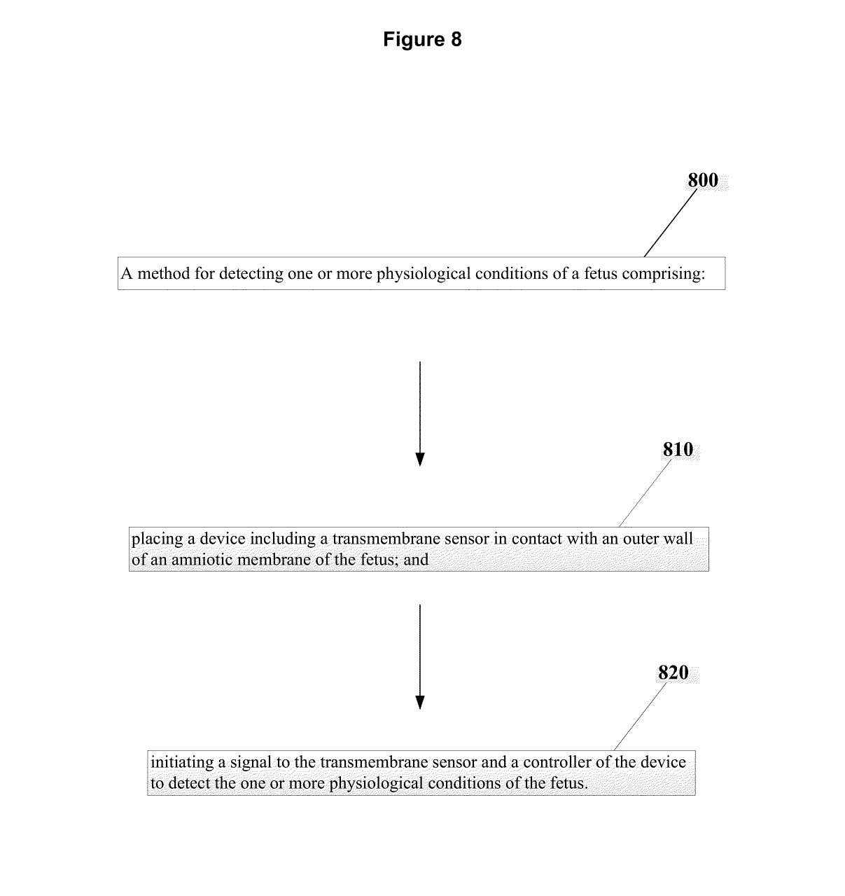

FIG. 8 illustrates an exemplary method 800 for detecting one or more physiological conditions of a fetus including placing 810 a transmembrane sensor in contact with an outer wall of an amniotic membrane of the fetus; and detecting 820 the one or more physiological conditions of the fetus from the transmembrane sensor.

### Sensors for Measuring Physiological Parameters in the Amniotic Fluid

The device includes one or more sensors for qualitatively and/or quantitatively measuring one or more physiological parameters in the amniotic fluid of a subject. The one or more sensors can include but are not limited to a biosensor, a chemical sensor, a physical sensor, an optical sensor, or a combination thereof. The one or more sensors can include one or more recognition elements that recognize one or more physiological parameters. The interaction of one or more physiological parameters with one or more sensors results in one or more detectable signals. Preferably the one or more sensors measure in real-time the levels of one or more physiological parameters in the amniotic fluid of a subject. Examples of physiological parameters include but are not limited to fetal tachycardia, respiratory distress syndrome, thyroid disease, body temperature, respiration rate, pulse, blood pressure, edema, oxygen saturation, pathogen levels, inflammatory mediators, cytokines, or toxin levels.

The one or more recognition elements that identify one or more physiological parameters in the amniotic fluid can include, but are not limited to, antibodies, antibody fragments, peptides, oligonucleotides, DNA, RNA, aptamers, protein nucleic acids proteins, viruses, enzymes, receptors, bacteria, cells, cell fragments, inorganic molecules, organic molecules, or combinations thereof. The one or more recognition elements can be associated with one or more substrate integrated into the one or more sensors.

The one or more sensors for sensing one or more physiological parameters can incorporate one or more recognition elements and one or more measurable fluorescent signal. In an embodiment, one or more physiological parameters in the amniotic fluid of a subject are captured by one or more recognition elements and further react with one or more fluorescent second elements. The fluorescence associated with the captured one or more physiological parameters can be measured using fluorescence spectroscopy. Alternatively, the fluorescence signal can be detected using at least one charged-coupled device (CCD) and/or at least one complimentary metal-oxide semiconductor (CMOS).

In an aspect, the one or more sensors can use Förster or fluorescence resonance energy transfer (FRET) to sense one or more physiological parameters in the amniotic fluid of a subject. FRET is a distance-dependent interaction between the electronic excited states of two dye molecules in which excitation is transferred from a donor molecule to an acceptor molecule without emission of a photon. In some aspects, interaction of a donor molecule with an acceptor molecule may lead to a shift in the emission wavelength associated with excitation of the acceptor molecule. In other aspects, interaction of a donor molecule with an acceptor molecule may lead to quenching of the donor emission. The one or more recognition elements associated with the one or more sensors may include at least one donor molecule and at least one acceptor molecule. Binding of one or more physiological parameters to the recognition element may result in a conformation change in the recognition element, leading to changes in the distance between the donor and acceptor molecules and changes in measurable fluorescence. The recognition element may be a cell, an antibody, an aptamer, a receptor or any other molecule that changes conformation or signaling in response to binding one or more physiological parameters.

A variety of donor and acceptor fluorophore pairs may be considered for FRET associated with the recognition element including, but not limited to, fluorescein and tetramethylrhodamine; IAEDANS and fluorescein; fluorescein and fluorescein; and BODIPY FL and BODIPY FL. A number of Alexa Fluor (AF) fluorophores (Molecular Probes-Invitrogen, Carlsbad, Calif., USA) may be paired with other AF fluorophores for use in FRET. Some examples include, but are not limited, to AF 350 with AF 488; AF 488 with AF 546, AF 555, AF 568, or AF 647; AF 546 with AF 568, AF 594, or AF 647; AF 555 with AF594 or AF647; AF 568 with AF6456; and AF594 with AF 647.

The cyanine dyes Cy3, Cy5, Cy5.5 and Cy7, which emit in the red and far red wavelength range (>550 nm), offer a number of advantages for FRET-based detection systems. Their emission range is such that background fluorescence is often reduced and relatively large distances (>100 Å) can be measured as a result of the high extinction coefficients and good quantum yields. For example, Cy3, which emits maximally at 570 nm and Cy5, which emits at 670 nm, may be used as a donor-acceptor pair. When the Cy3 and Cy5 are not proximal to one another, excitation at 540 nm results only in the emission of light by Cy3 at 590 nm. In contrast, when Cy3 and Cy5 are brought into proximity by a conformation change in an aptamer, antibody, or receptor, for example, excitation at 540 nm results in an emission at 680 nm. Semiconductor quantum dots (QDs) with various excitation/emission wavelength properties may also be used to generate a fluorescence based sensor.

Quenching dyes may be used as part of the binder element to quench the fluorescence of visible light-excited fluorophores. Examples include, but are not limited, to DABCYL, the non-fluorescing diarylrhodamine derivative dyes QSY 7, QSY 9 and QSY 21 (Molecular Probes, Carlsbad, Calif., USA), the non-fluorescing Black Hole Quenchers BHQ0, BHQ1, BHQ2, and BHQ3 (Biosearch Technologies, Inc., Novato, Calif., USA) and Eclipse (Applera Corp., Norwalk, Conn., USA). A variety of donor fluorophore and quencher pairs may be considered for FRET associated with the recognition element including, but not limited to, fluorescein with DABCYL; EDANS with DABCYL; or fluorescein with QSY 7 and QSY 9. In general, QSY 7 and QSY 9 dyes efficiently quench the fluorescence emission of donor dyes including blue-fluorescent coumarins, green- or orange-fluorescent dyes, and conjugates of the Texas Red and Alexa Fluor 594 dyes. QSY 21 dye efficiently quenches all red-fluorescent dyes. A number of the Alexa Fluor (AF) fluorophores (Molecular Probes-Invitrogen, Carlsbad, Calif., USA) may be paired with quenching molecules as follows: AF 350 with QSY 35 or DABCYL; AF 488 with QSY 35, DABCYL, QSY7 or QSY9; AF 546 with QSY 35, DABCYL, QSY7 or QSY9; AF 555 with QSY7 or QSY9; AF 568 with QSY7, QSY9 or QSY21; AF 594 with QSY21; and AF 647 with QSY 21.

The one or more sensor for sensing one or more physiological parameters can use the technique of surface plasmon resonance (for planar surfaces) or localized surface plasmon resonance (for nanoparticles). Surface plasmon resonance involves detecting changes in the refractive index on a sensor surface in response to changes in molecules bound on the sensor surface. The surface of the sensor may be a glass support or other solid support coated with a thin film of metal, for example, gold. The sensor surface may further carry a matrix to which is immobilized one or more recognition elements that recognize one or more physiological parameters. The one or more recognition elements that recognize one or more physiological parameters may be antibodies or fragments thereof, oligonucleotide or peptide based aptamers, receptors to physiological parameters or fragments thereof, artificial binding substrates formed by molecular imprinting, or any other examples of molecules and or substrates that bind physiological parameters. As amniotic fluid from the subject passes by the sensor surface, one or more physiological parameters may interact with one or more recognition elements on the sensor surface. The sensor is illuminated by monochromatic light. Resonance occurs at a specific angle of incident light. The resonance angle depends on the refractive index in the vicinity of the surface, which is dependent upon the concentration of molecules on the surface. An example of instrumentation that uses surface plasmon resonance is the BIACORE system (Biacore, Inc.—GE Healthcare, Piscataway, N.J.) which includes a sensor microchip, a laser light source emitting polarized light, an automated fluid handling system, and a diode array position sensitive detector. See, e.g., Raghavan & Bjorkman Structure 3: 331-333, 1995, which is incorporated herein by reference.

The one or more sensors can be one or more label-free optical biosensors that incorporate other optical methodologies, e.g., interferometers, waveguides, fiber gratings, ring resonators, and photonic crystals. See, e.g., Fan, et al., Anal. Chim. Acta 620: 8-26, 2008, which is incorporated herein by reference. For example, reflectometric interference spectroscopy can be used to monitor in real-time the interaction of the inflammatory mediator interferon 2 with an anti-interferon 2 antibody. See, e.g., Piehler & Schreiber, Anal. Biochem. 289: 173-186, 2001, which is incorporated herein by reference.

The one or more sensors for sensing one or more physiological parameters can be one or more microcantilevers. A microcantilever can act as a biological sensor by detecting changes in cantilever bending or vibrational frequency in response to binding of one or more physiological parameters to the surface of the sensor. In an aspect the sensor can be bound to a microcantilever or a microbead as in an immunoaffinity binding array. In another aspect, a biochip can be formed that uses microcantilever bi-material formed from gold and silicon, as sensing elements. See, e.g. Vashist J. Nanotech Online 3:DO: 10.2240/azojono0115, 2007, which is incorporated herein by reference. The gold component of the microcantilever can be coated with one or more recognition elements which upon binding one or more physiological parameters causes the microcantilever to deflect. Aptamers or antibodies specific for one or more physiological parameters can be used to coat microcantilevers. See, e.g., U.S. Pat. No. 7,097,662, which is incorporated herein by reference. The one or more sensor can incorporate one or more methods for microcantilever deflection detection including, but not limited to, piezoresistive deflection detection, optical deflection detection, capacitive deflection detection, interferometry deflection detection, optical diffraction grating deflection detection, and charge coupled device detection. In some aspects, the one or more microcantilever can be a nanocantilever with nanoscale components. The one or more microcantilevers and/or nanocantilevers can be arranged into arrays for detection of one or more physiological parameters. Both microcantilevers and nanocantilevers can find utility in microelectomechnical systems (MEMS) and/or nanoelectomechnical systems (NEMS) associated with an extracorporeal or intracorporeal device.

The one or more sensor for sensing one or more physiological parameters can be a field effect transistor (FET) based biosensor. In this aspect, a change in electrical signal is used to detect interaction of one or more analytes with one or more components of the sensor. See, e.g., U.S. Pat. No. 7,303,875, which is incorporated herein by reference.

The one or more sensors for sensing one or more physiological parameters can incorporate electrochemical impedance spectroscopy. Electrochemical impedance spectroscopy can be used to measure impedance across a natural and/or artificial lipid bilayer. The sensor can incorporate an artificial bilayer that is tethered to the surface of a solid electrode. One or more receptor can be embedded into the lipid bilayer. The one or more receptors can be ion channels that open and close in response to binding of a specific analyte. The open and closed states can be quantitatively measured as changes in impedance across the lipid bilayer. See, e.g., Yang, et al., IEEE SENSORS 2006, EXCO, Daegu, Korea/Oct. 22-25, 2006, which is incorporated herein by reference.

The one or more sensors for sensing one or more physiological parameters can be cells that include one or more binding elements which when bound to one or more physiological parameters induces a measurable or detectable change in the cells. The cells may emit a fluorescent signal in response to interacting with one or more physiological parameters. For example, a bioluminescent bioreporter integrated circuit may be used in which binding of a ligand to a cell induces expression of reporter polypeptide linked to a luminescent response. See, e.g., U.S. Pat. No. 6,673,596, and Durick & Negulescu Biosens. Bioelectron. 16: 587-592, 2001, which are incorporated herein by reference. Alternatively, the one or more cells may emit an electrical signal in response to interacting with one or more physiological parameters. In a further aspect, an implantable biosensor may be used which is composed of genetically-modified cells that responded to ligand binding by emitting a measurable electrical signal. See U.S. Patent Application 2006/0234369 A1, which is incorporated herein by reference.

A sensor on an external surface of the amniotic membrane or on the abdomen of the mother may be used to detect fetal lung maturation by means of measurement of the electrical conductivity of the amniotic fluid correlated with the phospholipid content in the amniotic fluid. A change may be observed in the electrical conductivity of the amniotic fluid in the last period of pregnancy, which reflects the increase in phospholipid concentration, when lung maturation is reached. See, e.g., Pachi et al., Fetal Diagn Ther, 16: 90-94, 2001, which is incorporated herein by reference.

### Transmembrane Therapeutic Drug Delivery Patch Across an Amniotic Membrane

The method for delivering a medicament to a fetus includes administering the medicament from an implantable patch configured to deliver the medicament across an amniotic membrane to amniotic fluid of the fetus. The implantable patch is adapted for delivery of one or more therapeutic compositions across the amniotic membrane for delivery of the one or more therapeutic compositions to the amniotic fluid and to the fetus. In some aspects, the patch includes a first layer comprising a biocompatible material, which is generally substantially drug-impermeable. The patch is placed in contact with a portion of an outer surface of an amniotic sac of the mother. When placed on the surface of the amniotic sac, the first layer and amniotic sac surface form a reservoir for the therapeutic composition, with the therapeutic composition in the reservoir in contact with the amniotic sac surface. Therapeutic composition in the reservoir traverses the tissue of the amniotic membrane surface to enter the amniotic fluid.

The implantable drug delivery patch may include a second layer in contact with the first layer, the second layer comprising a drug-permeable portion. In these embodiments, a reservoir for the therapeutic composition is defined by the first and second layers. The drug-permeable portion of the second layer is adjacent to the amniotic sac. Therapeutic composition exits the reservoir through the drug-permeable portion of the second layer, traverses the outer surface of the amniotic sac and the amniotic membrane, and enters the amniotic fluid.

The patch is affixed to the outer surface of the amniotic sac by an attachment element. The attachment element may be provided as an integral part of the first layer, or, if present, the second layer, or may be provided as a separate component which distinct from the second layer and which is affixed to the first layer or the second layer. The invention further relates to a drug delivery system comprising an implantable patch; a catheter having proximal and distal ends, the distal end being operably attached to the patch; and a drug delivery device operably attached to the proximal end of the catheter. The invention also provides methods for delivering a medicament to a fetus that includes administering a drug via a drug delivery system which comprises an implantable patch. In some aspects, the patch may include a refillable reservoir to contain the therapeutic composition. See, e.g., U.S. Pat. No. 6,726,920, which is incorporated herein by reference.

In some aspects the drug delivery patch may deliver the therapeutic composition across the amniotic sac into the amniotic fluid by one or more mechanisms including: iontophoresis; electroporation; application by ultrasound; or use of microscopic projection or microneedles. See, e.g., Patel et al., “Transdermal Drug Delivery System: A Review,” The Pharma Innovation, Volume 1, No. 4, pages 66-75, 2012, which is incorporated herein by reference.

The method for delivering a medicament to a fetus includes administering the medicament from an implantable patch including a microneedle array of polymeric materials configured to deliver the medicament across an amniotic membrane to amniotic fluid of the fetus. The microneedle patch of polymeric materials, includes a microneedle array and a support for the microneedles to stand and align upon. The microneedles are approximately 100-1000 μM in length, and have the ability to convert from hard solid state to hydrogel state by absorbing water. The microneedle have an ability to convert from hard solid state to hydrogel state by absorbing water from the tissues of the subject or from exogenously supplied water.

The microneedle system is formed of hydrophilic polymeric materials which are hard and strong enough to penetrate epidermis in a dry glassy state, but undergoes a phase-transition to hydrogel state by absorbing body fluid or exogenous water when in contact with dermis. This transdermal patch consists of a microneedle array and a drug reservoir plate (“holding plate”) on top of which the microneedles stand as an array (as an integrated piece). Therapeutics and other agents to be delivered can be loaded in the matrix of the needles and the reservoir plate, or loaded only in the needles. Therapeutics and other agents to be delivered can be loaded in layers or in alternating layers of therapeutic composition and non-therapeutic to provide timed dosage of the therapeutic composition.

The working mechanism of the phase-transition microneedle system is illustrated. The microneedles formed of the hydrophilic polymers penetrate the epidermis, then absorb body fluid to be hydrated to hydrogel state permeable to proteins, peptides, genes or other water soluble therapeutics loaded in the matrix of the needles and/or the reservoir plate. During the phase transition of the needles and the plate from dry state to hydrated gel state, diffusion channels for the lipophobic agents loaded in the system are opened (formed). This microneedle system differs from that made of polysaccharide in that the microneedles do not disappear by hydration, but remain in the skin as sustained diffusion channels. Controlled release delivery is achieved by three factors: polymer phase transition, drug diffusion, as well as the fabrication process of the microneedle patch (programmed casting).

In addition to the phase transition nature, one important advantage of this microneedle array system is its easy yet multi-functional fabrication process. The microneedle array can simply be prepared by casting an aqueous solution of the microneedle-forming polymer on a mold having microholes aligned on its surface as an array. The final form of the microneedle patch is formed by drying the casted solution and detached it from the mold. Drugs to be delivered are added into the polymer solution before casting on the mold. A unique and interesting feature of this system is that its fabrication process can be used to achieve a desired release pattern. By a programmed casting (i.e. casting polymer solutions with different drug concentration stepwise on the mold), a precisely programmed drug release profile can be achieved. In an aspect, the microneedle patches may be formed from polyvinyl alcohol (PVA) and dextran (PVA/dextran=80%/20%).

The polymeric materials for forming the microneedle array and plate are hydrophilic and soluble in water under certain condition but form water-insoluble hydrogel network by chemical cross-linking or by physical cross-linking The polymeric materials may be, for example, the combination of polyvinyl alcohol (PVA) and dextran, or PVA and chitosan, or PVA and alginate, or polyvinyl alcohol and hyaluronate, or PVA and polyethylene glycol (PEG). For example, the weight ratio of PVA/dextran is between 100/0 to 70/30, the weight ratio of PVA/chitosan is between 100/0 to 85/15, the weight ratio of PVA/alginate is between 100/0 to 85/15, the weight ratio of PVA/hyaluronate is 100/0 to 85/15, and the weight ratio of PVA/PEG is between 100/0 to 90/10. For example, the weight-average molecular weight of PVA is between 10,000-250,000, the weight-average molecular weight of dextran is between 6,000-5,000,000, the weight-average molecular weight of chitosan is between 20,000-4,000,000, the weight-average molecular weight of alginate is between 10,000-3,000,000, the weight-average molecular weight of hyaluronate is between 100,000-5,000,000, and the weight-average molecular weight of PEG is between 100-1,000. See, e.g., U.S. 2011/0195124, which is incorporated herein by reference.

### Microjet Delivery of Therapeutic Compositions Across the Amniotic Membrane to the Amniotic Fluid

A method for delivering a medicament to a fetus includes administering the medicament from a microjet injector containing a therapeutic composition placed at an amniotic membrane configured to deliver the medicament across the amniotic membrane to amniotic fluid of the fetus.

In an aspect, the method for delivering a medicament to a fetus can include administering the medicament from medicament applicators including one or more high speed microjets containing a therapeutic composition placed at an amniotic membrane configured to deliver the medicament across the amniotic membrane to amniotic fluid of the fetus. High speed microjets can deliver one or more medicaments by displacing the medicament solution through a micronozzle, e.g., 50-100 μm in final diameter. The high speed microjets can use one or more modes of fluid displacement, e.g., a piezoelectric actuator displacing a plunger, that provides a device or system having robustness and energy efficiency. The displacement of the plunger by the piezoelectric actuator can eject a microjet whose volume and velocity can be controlled by controlling the voltage and the rise time of the applied pulse to the piezoelectric actuator. At the end of the stroke, the plunger can be brought back to its original position by a compressed spring. The voltage applied to the piezoelectric crystal can be varied between 0 and 140 V to generate microjets with volumes up to 15 nanoliters. The frequency of pulses can be within a range of 0.1 to 10 Hz, e.g., 1 Hz. The medicament solution can be filled in a reservoir, which directly feeds the solution to the micronozzle of the microjet. The reservoir can be maintained at a slight overpressure, e.g., a small fraction of atmospheric pressure, to avoid backflow. In detailed aspects, the piezoelectric actuator, on application of a voltage pulse, can expand rapidly to push a plunger that ejects the fluid from the micronozzle as a high-speed microjet. The volume of the microjet is proportional to the amplitude of the voltage pulse, and the velocity of the microjet is proportional to the rise time. In further detailed aspects, a rise time of 10 μseconds would lead to a mean velocity of 127 meters/second for a 10-nanoliter microjet delivered from a 100-μm diameter micronozzle. For example, v=Q/At, where Q is the microjet volume, A is the cross-sectional area of the micronozzle, and t is the rise time. By controlling the amplitude and rise time of the pulse, velocity as well as volume of the microjet can be adjusted. Dispensed volume from the nozzle is replaced by liquid from the reservoir, which is maintained under slight positive pressure to avoid backflow. Under typical operating conditions, microjets can be ejected from the micronozzle at exit velocities exceeding 100 meters/second and volumes of 10 to 15 nanoliters. The microjets can be cylindrical in shape and each jet pulse could be clearly distinguished. To deliver volumes in excess of 10 to 15 nanoliters, the microjets can be designed to operate over a prolonged time period, and the total amount of liquid ejected will be proportional to the application time. In an aspect, a pulsation frequency of 1 Hz (1 microjet per second) can be used. This frequency can be increased if higher delivery rates are desired. See, e.g., Arora et al., Proc. Natl. Acad. Sci. USA, 104: 4255-4260, 2007, which is incorporated herein by reference. Other modes of fluid displacement from the high speed microjet include, but are not limited to, dielectric breakdown, electromagnetic displacement, springs, solenoids, motors, or compressed gas actuators.

In an aspect, the method for delivering a medicament to a fetus includes medicament applicators including one or more microneedles that can be produced by microfabrication technology. Microneedles can be used to deliver the one or more medicaments through the amniotic membrane to the amniotic fluid of the fetus. The microneedles pierce into the amniotic membrane to permit drug delivery, and are short and thin to avoid causing pain. The amniotic membrane of the fetus provides a barrier to drug transport into the amniotic fluid and to the body of the fetus. A microneedle can be configured to cross the amniotic membrane and deliver drugs into the amniotic fluid. Microneedles can be configured to pierce the amniotic membrane and to increase, by two or more orders of magnitude and over time, amniotic membrane permeability to small molecules and proteins. See, e.g., Kaushik et al., Anesth. Analg., 92: 502-504, 2001; Henry S, et al., J Pharm Sci., 87: 922-925, 1998; McAllister D, et al., Proc Int Symp Control Rel Bioact Mater., 26: 192-193, 1999, which are incorporated herein by reference. In an aspect, the device including medicament applicators can be microfabricated as one or more microfine lances, one or more microfine cannulas, or one or more microprojections.

In an aspect, the method for delivering a medicament to a fetus includes administering the medicament from an implantable patch including solid microneedles can be used in combination with transdermal patch technology configured to deliver the medicament across an amniotic membrane to amniotic fluid of the fetus. Integrated into a patch, microneedles can provide a minimally invasive method to increase amniotic membrane permeability for diffusion-based transport that could make transmembrane delivery of many drugs possible, including that of large molecules such as proteins. Hollow microneedles, either as individual needles or as multineedle arrays, can be used for convection-based delivery. This microinfusion approach can increase rates of delivery beyond those of passive patches, and permit rates to be modulated in real time by a microprocessor-controlled pump, which can include a user interface for input by healthcare providers. See, e.g., McAllister et al., Proc. Natl. Acad. Sci. USA, 100: 13755-13760, 2003, which is incorporated herein by reference.

In an aspect, the method for delivering a medicament to a fetus includes administering the medicament from an implantable patch with medicament applicators including one or more electrodes on microprojections configured to apply electrical energy to the amniotic membrane of the fetus. The electrodes on microprojections provide ablation of the amniotic membrane in an area beneath the electrodes thereby generating a plurality of hydrophilic microchannels in the amniotic membrane. The one or more medicaments can be delivered through the plurality of hydrophilic microchannels in the amniotic membrane. See, e.g., U.S. Pat. No. 7,395,111; or U.S. 2005/0226922, which are incorporated herein by reference.

Piezoelectric actuators can be configured to displace plungers in the high speed microjets. The piezoelectric actuators can include capacitive transducers that expand when voltage is applied to them. The displacements of piezoelectric actuators are typically small (e.g., typically less than 10 μm), while the forces they generate can be quite large, from approximately 1 N to approximately 1000 N. Typically, the expansion of a piezoelectric actuator is limited by size, but large displacements which result in larger velocities can be desirable. One way to amplify the motion of a piezoelectric actuator is to use flexural hinges. Expansion of the piezoelectric actuator in the horizontal direction (x-x) can lead to a push or pull of hinges in the vertical direction (y-y). See, e.g., U.S. 2008/0091139, which is incorporated herein by reference.

In an aspect, the method for delivering a medicament to a fetus includes administering the medicament from an implantable patch includes the applicator configured as a compressed gas actuator to pressurize the chamber for delivery of the one or more medicaments through one or more high speed microjets. A compressed gas actuator is necessary to pressurize the central aperture of the one or more high speed microjets. The compressed gas actuator can take the form of a gas canister linked to a button cylinder, with operation of the button cylinder releasing a fixed amount of gas, for example 5 ml, enabling the gas source to be used to deliver sequentially a plurality of discrete payloads of one or more medicaments without needing to be recharged. Alternatively, a closed gas cylinder containing a single dose of gas can be sufficient for a single medicament delivery from the one or more high speed microjets. The gas source can include, e.g., helium, with the gas cylinder containing helium gas at a pressure of between approximately 15 bar and approximately 35 bar, or around 30 bar. Helium, as a driver gas, can provide much higher gas velocity than air, nitrogen, or CO₂.

### Microcapsule Delivery of Therapeutic Compositions Across the Amniotic Membrane to the Amniotic Fluid

A method for delivering a medicament to a fetus includes administering the medicament from a transmembrane patch including microcapsules containing a therapeutic composition placed at an amniotic membrane configured to deliver the medicament across the amniotic membrane to amniotic fluid of the fetus. Microcapsules containing a therapeutic composition in combination with high-resolution ultrasonography provide the possibility of bypassing the placenta through direct intra-amniotic administration of the therapeutic composition by trans-amniotic membrane treatment. The principal advantages of direct treatment are the avoidance of maternal toxicity and the metabolic effects of administered agents and the obviation of concern about the lack of placental permeability and transfer.

Trans-amniotic membrane treatment that includes administration of the therapeutic composition across the amniotic membrane to treat fetal disease may be used for medical conditions affecting the fetus such as: Congenital adrenal hyperplasia (hydrocortisone or dexamethasone treatment and fludrocortisone treatment; methylmalonic acidemia (carnitine and cobalamin treatment); biotin-responsive multiple carboxylase deficiency (biotin treatment); cardiac arrhythmias (folic acid treatment); neural tube defects (folic acid treatment); fetal lung maturity (corticosteroid treatment). See, e.g., Evans et al., “Fetal Drug Therapy,” West. J. Med. 159: 325-332, 1993, which is incorporated herein by reference.

Methods for delivering a medicament to a fetus may include multi-compartment polymeric microcapsule drug delivery carriers containing a therapeutic composition. The polyelectrolyte multilayer multi-compartment microcapsule may be used for multi-functional diagnostics and drug delivery across the amniotic membrane to the fetus. Responsiveness of the polyelectrolyte multilayer multi-compartment microcapsule towards external stimuli, such as laser light, provides controlled and on-demand release of encapsulated therapeutic composition from the microcapsules. The external stimuli include light as a physical stimulus which has been widely used for activation of microcapsules and release of the therapeutic composition. Approaches exist to build multi-compartment microcapsules and to achieve controlled and triggered release from the sub-compartments using laser induced breakdown of sub-compartments to release the therapeutic composition. See, e.g., Xiong R, Soenen S J, Braeckmans K, Skirtach A G. “Towards Theranostic Multicompartment Microcapsules: in-situ Diagnostics and Laser-induced Treatment.” Theranostics, 3(3): 141-151, 2013, doi:10.7150/thno.5846, which is incorporated herein by reference.

Methods for delivering a medicament to a fetus may include in situ activation of microcapsules containing one or more therapeutic compositions may include the microcapsule containing two or more internal immiscible liquids enclosed together in a single polymer shell. A therapeutic composition precursor is associated with at least one internal liquid phase in the polymer shell. The microcapsule is exposed to an energy source in an amount effective to promote physical mixing of the immiscible liquid phases and to increase the activation kinetics of the therapeutic composition precursor. The energy source may be one or more of a source of ultraviolet light, an electromagnetic field, a radiofrequency, or microwave energy.

One of the internal liquid phases may an aqueous phase and the other of the internal liquid phases may be a hydrocarbon or oil phase. The aqueous phase and the hydrocarbon or oil phase may be in contact with and separated by a polymer membrane. The therapeutic composition precursor may be more soluble in the hydrocarbon or oil phase than in the aqueous phase. The activated therapeutic composition may be more soluble in the aqueous phase than in the hydrocarbon or oil phase. Alternatively, the therapeutic composition precursor may be more soluble in the aqueous phase than in the hydrocarbon or oil phase, and the activated therapeutic composition may be more soluble in the hydrocarbon or oil phase than in the aqueous phase. After the microcapsule has been delivered across the amniotic membrane and has entered the amniotic fluid, the internal aqueous phase and the internal hydrocarbon or oil phase in contact with the polymer membrane are subject to mixing upon rupture of the polymer membrane by the application of the energy source to the microcapsule, e.g., one or more of an energy source of ultraviolet light, electromagnetic energy, radiofrequency energy, or microwave energy. See, e.g., U.S. Pat. No. 6,099,864, which is incorporated herein by reference.

A method for delivering a medicament to a fetus may include light sensitive polymer-based microcapsules constructed using a layer-by-layer self-assembly method. The construction of light sensitive polymer-based microcapsules consists in absorbing oppositely charged polyelectrolytes onto charged sacrificial particles. Microcapsules display a broad spectrum of qualities over other existing microdelivery systems such as high stability, longevity, versatile construction and a variety of methods to encapsulate and release substances. Microcapsules may be utilized for encapsulation of materials and release of materials by light. Microcapsules may be made sensitive to light by incorporation of one or more of light sensitive polymers, functional dyes, or metal nanoparticles. Optically active substances may be inserted into the shell during the assembly as a polymer complex or following the shell preparation. Ultraviolet light-addressable microcapsules allow for remote encapsulation and release of materials. Visible light- and infrared light-addressable microcapsules offer a large array of release strategies for capsules, from approaches that include destructive capsules to highly sensitive reversible capsules. See, e.g., Bedard M F, De Geest B G, Skirtach A G, Mohwald H, Sukhorukov G B. “Polymeric microcapsules with light responsive properties for encapsulation and release.” Adv Colloid Interface Sci. 158(1-2): 2-14. Jul. 12, 2010; doi: 10.1016/j.cis.2009.07.007. Epub Aug. 3, 2009, which is incorporated herein by reference.

A method for delivering a medicament to a fetus may include administering the medicament from polyelectrolyte capsules having metal nanoparticles embedded in the walls of the capsule and a therapeutic composition enclosed within the capsule cavity. Capsules may be transferred across the amniotic membrane without release of the therapeutic composition upon transfer. Controlled release of the therapeutic composition may occur following transfer of the capsules across the amniotic membrane. Photoinduced heating of the metal nanoparticles in the capsule walls lead to rupture of the capsule walls, and release of the therapeutic composition into the amniotic fluid for therapeutic treatment of the fetus. The rate of opening of the capsule walls may be varied by the intensity of light transmitted to and received by the microcapsules. Capsule opening at moderate light intensities leads to release of a moderate amount of the therapeutic composition, whereas capsule opening at high light intensities leads to release of a relatively higher amount of the therapeutic composition. See, e.g., Munoz Javier A, del Pino P, Bedard M F, Ho D, Skirtach A G, Sukhorukov G B, Plank C, Parak W J. “Photoactivated release of cargo from the cavity of polyelectrolyte capsules to the cytosol of cells.” Langmuir. 24(21): 12517-12520, Nov. 4, 2008; doi: 10.1021/1a802448z. Epub Oct. 10, 2008, which is incorporated herein by reference.

Microcapsules may be loaded with therapeutic compositions into a reservoir in the device. Following delivery of the microcapsules across the amniotic membrane into the amniotic fluid, the microcapsules may be opened remotely and activated with a pulse of light. Alternatively the microcapsules may be opened remotely using biological triggers, such as a drop in blood sugar levels.

The method for delivering a medicament to a fetus may be used for treatment of one or more diseases, for example, in Table 1.

TABLE 1
Possible applications of a method for
delivering a medicament to a fetus.

| Disease | Treatment |
|---|---|
| Fetal arrhythmias | Treat fetus or mother with digoxin 1 mg |
| associated with | PO qid. |
| hydrops fetalis; | |
| Supraventricular | |
| tachycardias | |
| Congenital adrenal | Treat fetus or mother with |
| hyperplasia | dexamethasone, .25 mg PO qid continued |
| (masculinization of | until term. |
| female fetus) | |
| Thyrotoxicosis | Treat fetus or mother with |
| | propylthiouracil (300 mg/d PO and titrate |
| | dose). |
| Hypothyroidism: | Treat fetus by providing thyroid |
| | hormone; Treat fetus with L-thyroxine |
| | (500 mcg q2wk) Trans-amniotic. |
| Acidosis | Treat fetus by removing/transforming |
| | carbon dioxide, bicarbonate, lactic acid, |
| | and hydrogen ions. |
| Hypoxia: drugs which | Treat fetus by sensing “specific markers |
| assist in the | of cerebral injury“; |
| binding of oxygen | The fetal myocardium can also be |
| to fetal hemoglobin | protected from hypoxia; |
| | Treat fetus by using antioxidants and |
| | calcium antagonists such as Anipamil or |
| | ROS scavengers to protect against |
| | damage due to free radicals, e.g., calcium |
| | channel antagonists, cytokines, vitamins |
| | C and E or analogs (OPC-14117, MDL |
| | 74,722), melatonin, ascorbic and lipoic |
| | acids, polyphenols, and carotenoids, |
| | superoxide dismutase (SOD) and catalase |
| | (CAT), metal ion chelators, Glutathione, |
| | YM737, Creatine, or spin-trap |
| | scavenging. |
| Diabetes | Treat fetus with drugs which control |
| | insulin levels, hyperglycemia, fetal |
| | assymetric overgrowth. |
| Congenital diseases: | Treat fetus with gene therapy to correct |
| hemoglobinopathies | congenital defects. |
| (e.g., sickle cell | |
| disease, | |
| thalassemias), | |
| immunodeficiency | |
| diseases, inborn | |
| errors of metabolism | |
| Methylmalonic | Treat fetus with prenatal cyanocobalamin |
| acidemia | administered orally to mother; titrate to |
| | high maternal plasma B12 levels. |
| Lung maturity | Treat fetus or mother with betamethasone |
| induction to | (12 mg IM q24h for 2 doses) for fetuses |
| prevent/reduce | at risk of preterm delivery (<34 weeks |
| respiratory | gestation). |
| distress syndrome | |

The method for delivering a medicament to a fetus may be used to deliver therapeutic agents to the fetus by administering the medicament across the amniotic membrane to amniotic fluid of the fetus from an injector non-invasively positioned to the amniotic fluid. Instillation of antibiotics, thyroxine, nutrients (i.e., dextrose, amino acids, and lipids), glucocorticoids, growth factors, surfactants, and β-adrenergic-receptor agonists directly into the amniotic fluid for delivery to the fetal circulation by administering the medicament across an amniotic membrane to amniotic fluid of the fetus from an injector non-invasively positioned to the amniotic fluid at the amniotic membrane. The medicament may be administered across the amniotic membrane to the amniotic fluid for delivery to the fetal circulation by either fetal swallowing or via the intramembranous route across non-keratinized epithelium of the fetus. See, e.g., Underwood et al., “State of the Art, Amniotic Fluid: Not Just Fetal Urine Anymore,” J. Perinatology 25: 341-348, 2005 which is incorporated herein by reference.

The method for delivering a medicament to a fetus that includes medicament administered to the amniotic fluid indicates that the amniotic cavity offers a unique mode of delivery of therapeutic agents to the fetus, via both fetal swallowing and the intramembranous route to the fetus. The method includes administering the medicament across an amniotic membrane to amniotic fluid of the fetus from an injector non-invasively positioned to the amniotic fluid at the amniotic membrane.

In clinical obstetrics, abnormalities of amniotic fluid volume occur commonly and often have unknown etiologies. Oligohydramnios occurs in 8.2% and polyhydramnios in 1.6% of all pregnancies prior to labor, with oligohydramnios in as many as 37.8% of laboring women. These volume disturbances are associated with significantly increased perinatal morbidity compared with outcomes when AF volume is within the normal range. Oligohydramnios and polyhydramnios may represent either a primary cause of fetal morbidity or a secondary effect of fetal abnormalities. As a primary cause, oligohydramnios beginning during the first or second trimester may compress the fetus and induce a series of structural and anatomical malformations (oligohydramnios deformation syndrome), which are often associated with in utero or neonatal death. During the late second or third trimester, a reduction in AF volume may be associated with umbilical cord compression, resulting in fetal heart rate decelerations, operative deliveries and/or intrauterine fetal demise. Alternatively, oligohydramnios may occur secondary to fetal renal obstruction or renal agenesis and perhaps chronic fetal hypoxemia. Polyhydramnios may directly result in maternal respiratory compromise from uterine overdistension, preterm labor, premature rupture of membranes, fetal malposition, umbilical cord prolapse and/or postpartum uterine atony. Further, polyhydramnios may be secondarily associated with a variety of fetal structural anomalies (e.g., esophageal atresia, anencephaly), cardiac arrhythmias, congenital infections or chromosomal abnormalities.

Medicaments administered directly into the amniotic fluid for delivery to the fetal circulation across an amniotic membrane to amniotic fluid of the fetus from an injector non-invasively positioned to the amniotic fluid at the amniotic membrane may be therapy for polyhydramnios or oligohydramnios that includes indomethacin for polyhydramnios; maternal and/or fetal 1-deamino-(8-D-arginine) vasopressin or desmopressin DDAVP®) for oligohydramnios; amniocentesis for twin pregnancies with oligohydramnios and polyhydramnios; amnioinfusion for oligohydramnios and occlusive AF catheters for ruptured membranes. Ultrasound determination of oligohydramnios may be used to determine candidates for saline amnioinfusion. Restoration of the AF volume with saline amnioinfusion for oligohydramnios and meconium in AF may be effective in reducing the requirement for Cesarean delivery for patients with intrapartum fetal heart rate abnormalities.

A method for delivering a medicament to a fetus that includes intra-amniotic fetal therapy may be highly efficient and effective. Thyroxine administration administered across the amniotic membrane to amniotic fluid of the fetus may reverse fetal hypothyroidism.

Treatments for intra-uterine growth restriction (IUGR) by administration of a medicament across the amniotic membrane to amniotic fluid of the fetus include therapeutic treatment of amniotic fluid and the fetus with dextrose, amino acids and lipids, or a combination of these. The therapeutic medicaments are administered across an amniotic membrane to amniotic fluid of the fetus from an injector non-invasively positioned to the amniotic fluid at the amniotic membrane.

The method for delivering a medicament to a fetus may include administration of a medicament across the amniotic membrane to amniotic fluid of the fetus, wherein the medicaments include, e.g., thyroid hormones, surfactants, glucocorticoids, β-adrenergic-receptor agonists, epidermal growth factors and purine nucleotides. The method may include treatment with a medicament such as surfactant to treat respiratory distress syndrome (RDS) in the fetus.

The method for delivering a medicament to a fetus may include administration of a medicament across the amniotic membrane to amniotic fluid of the fetus by gene therapy, in particular for the treatment of single gene defects, by administering the gene therapy vector across an amniotic membrane to amniotic fluid of the fetus from an injector for the gene therapy vector non-invasively positioned to the amniotic fluid at the amniotic membrane.

Four criteria for a method for delivering a medicament to a fetus that includes administration of a medicament across the amniotic membrane to amniotic fluid of the fetus are: First, the treatment should be specific for a given condition; Second, a target organ should be established; Third, the intra-amniotic therapy should be based on physiologically confirmed routes of amniotic fluid movement in order for the agent to reach its target destination; Finally, for specific fetal disorders, normal values of amniotic fluid, intramembranous and placental fluxes and pharmacokinetics should be established.

As alterations in maternal hydration produce changes in amniotic fluid dynamics, maternal plasma osmolality and fetal amniotic fluid osmolality may be therapeutically altered to modify fetal and amniotic fluid status. A method for delivering a medicament to a fetus includes an approach using maternal desmopressin (DDAVP®) and/or fetal DDAVP administered across an amniotic membrane to amniotic fluid of the fetus, plus water ingestion therapy for treatment of oligohydramnios. The antidiuretic action of DDAVP minimizes maternal loss of the hydrating fluid, thereby creating maternal and fetal hypo-osmolality. The fetus responds by increasing urine output and amniotic fluid volume. Fetal swallowing activity is also suppressed by plasma hypotonicity, a response that should aid the expansion of the amniotic fluid volume. See, e.g., M. G. Ross, et al, “National Institute of Child Health and Development Conference summary: Amniotic fluid biology—basic and clinical aspects,” The Journal of Maternal-Fetal Medicine, 10: 2-19, 2001, which is incorporated herein by reference.

### Prophetic Exemplary Embodiments

### EXAMPLE 1

Delivery of Digoxin Across the Amniotic Membrane to a Fetus with Tachyarrhythmia.

Medications are delivered across the amniotic sac to treat fetal tachycardia (abnormal accelerated heart rate). Ultrasound imaging suggests a 28 week-old fetus has hydrops fetalis (excess fluid accumulation in the fetus) and echocardiography detects tachycardia with a heart rate greater than 180 beats/minute. To treat tachycardia a drug delivery patch is surgically implanted on the outermost side of the amniotic sac to safely deliver digoxin (a cardiac glycoside) or a combination of flecainide and digoxin directly to the fetus.

A drug delivery patch which contains digoxin or a combination of flecainide and digoxin is implanted on the external (outermost) surface of the amniotic sac. See FIG. 1. The patch is constructed with a backing membrane, a reservoir containing digoxin, a drug permeable membrane and an adhesive which adheres to the amniotic sac and releases digoxin across the amniotic sac membranes into the amniotic fluid over an extended period. The patch may be surgically implanted on the amniotic sac and deliver digoxin transmembrane to the amniotic fluid for days or weeks (see e.g., Patel et al., “Transdermal Drug Delivery System: A Review,” The Pharma Innovation, Volume 1, No. 4, pages 66-75, 2012, and U.S. Pat. No. 6,726,920 issued to Theeuwes et al. on Apr. 27, 2004 which are incorporated herein by reference). For example a drug delivery patch may be fabricated with: (1) an outer layer or backing of polyurethane which is impervious to digoxin and body fluids; (2) a membrane of polypropylene which is permeated by digoxin and (3) an adhesive (e.g., a thrombin-based sealant, Vitex, available from V.I. Technologies, NY). Digoxin is formulated with a penetration agent (e.g., cyclohexanol) to promote penetration of digoxin across the amniotic sac into the amniotic fluid. The polypropylene membrane and penetration agent allow a controlled rate of digoxin delivery to the amniotic fluid. For example, digoxin may be released from the patch to sustain a therapeutic concentration of approximately 2 ng/mL in the amniotic fluid (see e.g., Jaeggi et al., Circulation 124: 1747-1754, 2011 which is incorporated herein by reference). Since amniotic fluid volumes can vary between approximately 25 mL and 800 mL depending on gestational age (Underwood et al., “State of the Art, Amniotic Fluid: Not Just Fetal Urine Anymore,” J. Perinatology 25: 341-348, 2005, which is incorporated herein by reference), the dose and schedule of digoxin release may be adjusted according to gestational age. For example, a 28 week fetus may have an amniotic fluid volume of approximately 800 mL which requires 1600 ng of digoxin delivered daily to maintain a concentration of 2 ng/ml, assuming depletion of digoxin and turnover of amniotic fluid each day. Thus a drug delivery patch with a total of 80 μg of digoxin can treat a fetus for 50 days. In contrast, oral dosing of the mother would require approximately 1 milligram per day of digoxin to maintain a therapeutic level of 2 ng/ml (see e.g., Jaeggi et al., Ibid.) with the attendant risks to the mother (see e.g., Springer, “Fetal Therapy: Options and Medical Treatment” available online at http://emedicine.medscape.com/article/936318-overview#aw2aab6b7 which is incorporated herein by reference). The drug delivery patch may also have a catheter entering the drug reservoir to supply additional digoxin to the reservoir. The catheter leads from the patch to the external surface of the mothers abdomen where a sterile port for injecting or withdrawing digoxin is implanted (see e.g., U.S. Pat. No. 6,726,920, Ibid.). Based on echocardiography and the fetal heart rate the dose and schedule of digoxin in the reservoir of the patch may be adjusted. For example, additional digoxin solution may be added to the reservoir to extend digoxin dosing beyond 50 days.

The digoxin may be enclosed in microcapsules in the drug delivery patch reservoir. The digoxin-containing microcapsules enter the amniotic fluid across the amniotic membrane. The digoxin enters the amniotic fluid in a timed-release manner or a delayed-release manner from the digoxin-containing microcapsules. The digoxin may be released by dissolution of the microcapsule or by utilizing sonophoresis or iontophoresis at the amniotic membrane to break down the microcapsules once the microcapsules have entered the amniotic fluid.

The digoxin-containing microcapsules in the drug delivery patch reservoir enter the amniotic fluid across the amniotic membrane. The microcapsules may be light-activated to release the digoxin into the amniotic fluid. The light source is implanted subcutaneous to the maternal skin and is located in contact with an outside surface of the amniotic membrane. Alternatively, the light source may be attached to an external surface of the maternal skin.

Multicompartment polymeric microcapsule drug delivery carriers include polyelectrolyte multilayer capsules that may be used for multi-functional diagnostics and drug delivery of digoxin across the amniotic membrane to the amniotic fluid and to the fetus. The multicompartment polymeric microcapsule are responsive towards external stimuli, such as laser light, and provide controlled and on-demand release of encapsulated digoxin from the microcapsule. The external stimuli, including light as a physical stimulus, has been used for activation of microcapsules and release of the digoxin therapeutic into the amniotic fluid. Approaches exist to build multicompartment microcapsules and to achieve controlled and triggered release from their subcompartments using laser induced breakdown of subcompartments to release digoxin therapeutic. See, e.g., Xiong R, Soenen S J, Braeckmans K, Skirtach A G. “Towards Theranostic Multicompartment Microcapsules: in-situ Diagnostics and Laser-induced Treatment.” Theranostics, 3(3): 141-151, 2013, doi:10.7150/thno.5846, which is incorporated herein by reference.

In situ activation of microcapsules containing one or more therapeutic compositions may include microcapsules that contain two or more internal immiscible liquids enclosed together in a single polymer shell. A therapeutic drug precursor is associated with at least one internal liquid phase. The microcapsule is exposed to an energy source in an amount effective to promote physical mixing of the immiscible liquid phases and to increase the activation kinetics of the therapeutic drug precursor. The energy source may be one or more of a source of ultraviolet light, an electromagnetic field, a radiofrequency, or microwave energy.

One of the internal liquid phases may an aqueous phase and the other of the internal liquid phases may be a hydrocarbon or oil phase. The aqueous phase and the hydrocarbon or oil phase may be in contact with the polymer membrane. The drug precursor may be more soluble in the hydrocarbon or oil phase than in the aqueous phase. The activated drug may be more soluble in the aqueous phase than in the hydrocarbon or oil phase. Alternatively, the drug precursor may be more soluble in the aqueous phase than in the hydrocarbon or oil phase, and the activated drug may be more soluble in the hydrocarbon or oil phase than in the aqueous phase. After the microcapsule has been delivered across the amniotic membrane and has entered the amniotic fluid, the internal aqueous phase and the internal hydrocarbon or oil phase in contact with the polymer membrane are subject to mixing by the application of the energy source to the microcapsule after the microcapsule has entered the amniotic fluid. The active therapeutic drug is released into the amniotic fluid. See, e.g., U.S. Pat. No. 6,099,864, which is incorporated herein by reference.

### EXAMPLE 2

### Prevention of Respiratory Distress Syndrome by Delivery of Corticosteroid to Amniotic Fluid.

Corticosteroids are delivered across the amniotic sac to induce fetal lung maturation in a fetus at risk of preterm delivery. A fetus at 33 weeks gestation is treated with a patch which is attached to the outermost side of the amniotic sac to safely deliver corticosteroids directly to the fetus. The patch contains an array of degradable microneedles to penetrate the amniotic sac and deliver corticosteroid to the amniotic fluid.

A drug delivery patch with an array of microneedles which contain a corticosteroid, betamethasone, is implanted on the external (outermost) surface of the amniotic sac. See FIG. 1. The drug delivery patch is attached to the amniotic sac with an adhesive to keep it in place with the microneedles penetrating the amniotic sac (see e.g., U.S. Patent Application 2011/0195124 by Jin published on Aug. 11, 2011, which is incorporated herein by reference). The patch may be implanted on the amniotic sac by minimally invasive methods, e.g., laparoscopy (see e.g., U.S. Pat. No. 6,726,920 issued to Theeuwes et al. on Apr. 27, 2004 which is incorporated herein by reference). The drug delivery patch may be biodegradable thus avoiding the need to surgically remove it. Biodegradable patches are described (see e.g., U.S. Pat. No. 6,726,920, Ibid.). The patch is constructed with microneedles containing betamethasone which penetrate the amniotic sac and release betamethasone into the amniotic fluid over a period of approximately 48 hours. The microneedles are approximately 20.0 μm in length in order to penetrate into the amniotic membrane layer (see e.g., Bourne, Postgrad. Med. J. 38: 193-201, 1962 and Fetterolf et al., Wounds 24: 299-307, 2012 which are incorporated herein by reference). FIG. 2 shows a model of an amniotic sac with microneedles penetrating the chorion layer, the intermediate layer and the amnion layer. The microneedles, comprised of polymers and containing betamethasone, convert from a rigid needle structure to a hydrogel once inserted in the amniotic sac and release betamethasone into the amniotic fluid. Polymeric, degradable microneedle arrays may be designed with preferred release kinetics (see e.g., U.S. Patent Application No. 2011/0195124, Ibid.). Betamethasone is released from the microneedles over 48 hours (see e.g., Garite et al., Am. J. Obstet. Gynecol. 200: 248e1-248e9, 2009 which is incorporated herein by reference.). A 33 week fetus may have an amniotic fluid volume of approximately 800 mL which may require delivery of approximately 24 micrograms of betamethasone to achieve an amniotic fluid concentration of 30 nanogram/mL. Thus a drug delivery patch with a total of 48 micrograms of betamethasone which is released over 48 hours is implanted on the amniotic sac. In contrast, equivalent dosing by intramuscular injection of the mother would require approximately two 12 milligram doses of betamethasone 24 hours apart (see e.g., Petersen et al., Br. J. Clin. Pharmac. 18: 383-392, 1984 which is incorporated herein by reference). Increased susceptibility to infections and depression of systemic cortisol levels are a few of the negative side effects suffered by pregnant mothers given corticosteroids. Direct delivery of corticosteroids to the fetus via the amniotic fluid avoids systemic effects to the mother and allows better control of corticosteroid dosage to the fetus.

A drug delivery patch with an array of microjets which contain a corticosteroid, betamethasone, is implanted on the external (outermost) surface of the amniotic sac. The microjets are connected to a reservoir containing betamethasone. The drug delivery patch is attached to the amniotic sac with an adhesive to keep it in place with the microjets for delivering corticosteroid across the amniotic sac (see e.g., U.S. Patent Application 2011/0195124 by Jin published on Aug. 11, 2011 which is incorporated herein by reference). The patch may be implanted on the amniotic sac by minimally invasive methods, e.g., laparoscopy (see e.g., U.S. Pat. No. 6,726,920 issued to Theeuwes et al. on Apr. 27, 2004 which is incorporated herein by reference). Piezoelectric crystal actuators expand when electronically activated to drive small liquid volumes, e.g., 10-15 nanoliters, through a microjet at high velocity, greater than 100 meter/sec. See, e.g., U.S. 2008/0091139 entitled, “Methods, Devices and Kits for Microjet Drug Delivery” published Apr., 17, 2008; and Arora et al., Ibid., which is incorporated herein by reference. The rate of drug delivery may be modulated by varying the electronic signal frequency. For example, electronic signals to piezoelectronic crystals at 10 Hz actuate microjets ten times faster than 1 Hz signals and increase the rate of drug delivery by ten-fold. See, e.g., Arora et al., Ibid. In addition, the dosing of betamethasone may also be controlled by variation of the drug concentration in the reservoir. A drug delivery patch with a microjet array having a total of 48 micrograms of betamethasone in a reservoir which is released over 48 hours is implanted on the amniotic sac.

### EXAMPLE 3

### Method for Treating Autoimmune Thyroid Disease by Delivery of Thyroxine to Amniotic Fluid Using an Implanted Patch and Ultrasound.

A pregnant woman with autoimmune thyroid disease is treated with an implanted patch which includes liposomes that contain L-thyroxine. L-thyroxine is delivered from the patch across amniotic sac membranes using sonophoresis. Intra-amniotic delivery of L-thyroxine is necessary since L-thyroxine does not efficiently pass through the placenta (see e.g., Evans, et al., West. J. Med. 159: 325-332, 1993 which is incorporated herein by reference).

A patch is implanted on the outer surface of the amniotic sac to provide L-thyroxine to a fetus with a hypothyroid mother. The patch is constructed with a backing membrane, a reservoir holding gas liposomes containing L-thyroxine, a polymer responsive to ultrasound (US) and an adhesive which adheres to the amniotic sac (see e.g., Patel et al., “Transdermal Drug Delivery System: A Review,” The Pharma Innovation, volume 1, No. 4, pages 66-75, 2012; Norris et al., Antimicrobial Agents and Chemotherapy 49: 4272-4279, 2005, and U.S. Pat. No. 6,726,920 issued to Theeuwes et al. on Apr. 27, 2004 which are incorporated herein by reference). The patch may be surgically implanted on the amniotic sac and deliver L-thyroxine transmembrane across the amniotic membrane to the amniotic fluid in response to ultrasound applied from an external transducer (see e.g., U.S. Pat. No. 6,842,641 issued to Weimann et al. on Jan. 11, 2005 which is incorporated herein by reference). For example a drug delivery patch may be fabricated with: (1) an outer layer or backing of polyurethane which is impervious to L-thyroxine and body fluids; (2) a reservoir to hold gas liposomes, (3) an inner layer or membrane of poly(2-hydroxyethyl methacrylate hydrogel and (4) an adhesive (e.g., a thrombin-based sealant, Vitex, available from V.I. Technologies, NY). Gas liposomes undergo cavitation in response to ultrasound and promote transmembrane delivery. See e.g., Pitt et al., Expert Opinion Drug Delivery 1: 37-56, 2004 and Rao and Nanda, Journal Pharmacy and Pharmacology 61: 689-705, 2009 which are incorporated herein by reference. To initiate L-thyroxine delivery into the amniotic fluid an external ultrasound transducer is focused on the patch to disrupt the ultrasound labile inner membrane of the patch which is permeabilized by exposure to low-intensity ultrasound, approximately 43 kHz (see e.g., Norris et al., Ibid.). Also acoustically active liposomes containing L-thyroxine, (comprised of oil, phospholipids, and perfluorobutane gas; see e.g., Pitt et al., Ibid.) are irradiated with ultrasound to promote transmembrane delivery of L-thyroxine. Ultrasound irradiation is stopped after approximately 20 minutes and the inner membrane of the patch reestablishes a barrier to drug flow. Repeated irradiation with ultrasound is used to initiate delivery of multiple doses of L-thyroxine to the amniotic fluid. The fetus is monitored with ultrasound imaging, with special attention to goiter and morphometry associated with thyroid disease. L-thyroxine may be delivered repeatedly until term if necessary. The patch may be recovered after delivery.

Light sensitive polymer-based microcapsules containing L-thyroxine may be constructed using a layer-by-layer self-assembly method, which consists in absorbing oppositely charged polyelectrolytes onto charged sacrificial particles. Microcapsules display a broad spectrum of qualities over other existing microdelivery systems such as high stability, longevity, versatile construction and a variety of methods to encapsulate and release substances. Microcapsules may be utilized for encapsulation of L-thyroxine and release of L-thyroxine by light. Microcapsules may be made sensitive to light by incorporation of light sensitive polymers, functional dyes and metal nanoparticles. Optically active substances may be inserted into the shell during their assembly as a polymer complex or following the shell preparation. Ultraviolet-addressable microcapsules are shown to allow for remote encapsulation and release of materials. Visible- and infrared-addressable microcapsules offer a large array of release strategies for capsules, from destructive to highly sensitive reversible approaches. See, e.g., Bédard M F, De Geest B G, Skirtach A G, Möhwald H, Sukhorukov G B. “Polymeric microcapsules with light responsive properties for encapsulation and release.” Adv Colloid Interface Sci. 158(1-2): 2-14. Jul. 12, 2010; doi: 10.1016/j.cis.2009.07.007. Epub Aug. 3, 2009, which is incorporated herein by reference.

Microcapsules may be loaded with L-thyroxine into a reservoir in the device. The microcapsules are delivered across the amniotic membrane into the amniotic fluid. The microcapsules are opened remotely using biological triggers, such as a drop in blood sugar levels, or activated manually with a pulse of light.

The device including the patch may further include one or more sensors using fluorescence resonance energy transfer (FRET) to sense levels of thyroid hormone in the amniotic fluid of a subject. FRET is a distance-dependent interaction between the electronic excited states of two dye molecules in which excitation is transferred from a donor molecule to an acceptor molecule without emission of a photon. In some aspects, interaction of a donor molecule with an acceptor molecule may lead to a shift in the emission wavelength associated with excitation of the acceptor molecule. In other aspects, interaction of a donor molecule with an acceptor molecule may lead to quenching of the donor emission. The one or more recognition elements associated with the one or more sensors may include at least one donor molecule and at least one acceptor molecule. Binding of thyroid hormone from the amniotic fluid to the recognition element may result in a conformation change in the recognition element, leading to changes in the distance between the donor and acceptor molecules and changes in measurable fluorescence depending upon the concentration of thyroid hormone in the amniotic fluid. The recognition element may be an antibody, an aptamer, or a receptor to thyroid hormone that changes conformation or signaling in response to binding thyroid hormone.

To monitor thyroxine levels in amniotic fluid an optical sensor device may be incorporated in the patch to sample and analyze amniotic fluid and to provide feedback to guide dosing of thyroxine. For example a sensor to detect thyroxine may be a small self-contained optical sensor (see e.g., U.S. Pat. No. 6,304,766 issued to Colvin, Jr. on Oct. 16, 2001 which is incorporated herein by reference). The sensor is constructed as a cylindrical or capsular shape, approximately 500 microns to 2000 microns in length and approximately 300 microns in diameter and includes a light emitting diode (LED) to irradiate the fluorescent sensor protein which is attached to the exterior of the sensor body. For example FRET sensor protein comprised of two fluorescent proteins fused to a thyroxine (T4) recognition element may be used to detect T4. FRET sensors for metabolites and hormones may include a recognition element sandwiched between two fluorescent proteins, e.g., a yellow fluorescent protein, Venus, and cyan fluorescent protein, mTFP (see e.g., San Martin et al., (2013) PLoS ONE 8: e57712 doi:10.1371/journal.pone.0057712 which is incorporated herein by reference). Irradiation of the FRET sensor with 430 nm wavelength light results in emissions at 492 nm and 526 nm from mTFP and Venus, respectively. Binding of analyte, e.g., T4, to the recognition element may change the conformation of the sensor protein and change the fluorescence resonant energy transfer between the fluorescent proteins. Analyte concentrations as low as 36 picomoles/liter and as high as 10 millimoles/liter may be measured with a FRET sensor. See e.g., Nguyen et al., Adv. Nat. Nanosci. Nanotechnol. 3: 035011, September, 2012; http://dx.doi.org/10.1088/2043-6262/3/3/035011 and San Martin et al., Ibid. which are incorporated herein by reference. The optical sensor device has a body matrix formed from a transparent polymer (e.g., polymethylmethacrylate) to allow transmission of light from the LED to the fluorescent proteins and to the photodetector which detects fluorescent emissions. The T4 FRET sensor protein, comprised of a T4 recognition element, e.g., binding protein, or receptor, sandwiched between two fluorescent proteins, may be attached to the outer surface of the microsensor. The optical sensor device may also have a micropump and reservoir with saline to wash the FRET sensor protein and renew the T4 recognition element prior to making T4 measurements. See e.g., San Martin et al., Ibid. and U.S. Pat. No. 6,304,766 Ibid. The optical sensor device also has microcircuitry to transmit the data on T4 concentration in the amniotic fluid to a remote computer or mobile device, e.g., cell phone, tablet, or laptop, and inform a caregiver for T4 dosing. Data from periodic measurements of T4 concentrations may be used to adjust the dose and schedule of T4 delivery from the patch.

Each recited range includes all combinations and sub-combinations of ranges, as well as specific numerals contained therein.

All publications and patent applications cited in this specification are herein incorporated by reference to the extent not inconsistent with the description herein and for all purposes as if each individual publication or patent application were specifically and individually indicated to be incorporated by reference for all purposes.

Those having ordinary skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having ordinary skill in the art will recognize that there are various vehicles by which processes and/or systems and/or other technologies disclosed herein can be effected (e.g., hardware, software, and/or firmware), and that the preferred vehicle will vary with the context in which the processes and/or systems and/or other technologies are deployed. For example, if a surgeon determines that speed and accuracy are paramount, the surgeon may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies disclosed herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those having ordinary skill in the art will recognize that optical aspects of implementations will typically employ optically-oriented hardware, software, and or firmware.

In a general sense the various aspects disclosed herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices disclosed herein, or a microdigital processing unit configured by a computer program which at least partially carries out processes and/or devices disclosed herein), electrical circuitry forming a memory device (e.g., forms of random access memory), and/or electrical circuitry forming a communications device (e.g., a modem, communications switch, or optical-electrical equipment). The subject matter disclosed herein may be implemented in an analog or digital fashion or some combination thereof.

At least a portion of the devices and/or processes described herein can be integrated into a data processing system. A data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

The herein described components (e.g., steps), devices, and objects and the description accompanying them are used as examples for the sake of conceptual clarity and that various configuration modifications using the disclosure provided herein are within the skill of those in the art. Consequently, as used herein, the specific examples set forth and the accompanying description are intended to be representative of their more general classes. In general, use of any specific example herein is also intended to be representative of its class, and the non-inclusion of such specific components (e.g., steps), devices, and objects herein should not be taken as indicating that limitation is desired.

With respect to the use of substantially any plural or singular terms herein, the reader can translate from the plural to the singular or from the singular to the plural as is appropriate to the context or application. The various singular/plural permutations are not expressly set forth herein for sake of clarity.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

While particular aspects of the present subject matter described herein have been shown and described, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an”; the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for delivering a medicament to a fetus comprising:
administering the medicament across an amniotic membrane to amniotic fluid of the fetus from an injector non-invasively positioned to the amniotic fluid.

2. The method of claim 1, comprising administering the medicament from the injector surgically emplaced at an outer wall of the amniotic membrane.

3. The method of claim 2, comprising administering the medicament by one or more of microneedle, microjet, microcapsules, iontophoresis, and sonophoresis.

4. The method of claim 2, comprising laproscopically placing the injector at the outer wall of the amniotic membrane.

5. The method of claim 2, wherein the injector is configured to be placed laproscopically at the outer wall of the amniotic membrane.

6. The method of claim 2, wherein the injector is configured to be placed for one-time delivery.

7. The method of claim 1, placing the injector includes a transmembrane patch at an amniotic membrane and configuring the transmembrane patch to deliver the medicament across the amniotic membrane to the amniotic fluid of the fetus.

8. The method of claim 7, wherein the transmembrane patch is configured to deliver the medicament by one or more of microneedle, microjet, microcapsules, iontophoresis, and sonophoresis.

9. The method of claim 7, wherein the transmembrane patch is configured to deliver the medicament in response to a delivery schedule.

10. The method of claim 7, wherein the transmembrane patch is configured to deliver the medicament in response to an external command.

11. The method of claim 1, comprising placing a sensor configured to sense one or more physiological conditions in proximity to the fetus.

12. The method of claim 11, comprising initiating a signal to control administration of the medicament regulated by a controller in response to the one or more sensed physiological conditions.

13. The method of claim 1, comprising administering the medicament at maternal epithelium by one or more of microjets and microneedles.

14. The method of claim 1, comprising administering the medicament at maternal epithelium by microcapsules.

15. The method of claim 15, comprising administering the medicament at maternal epithelium by one or more of iontophoresis and sonophoresis.

16. The method of claim 1, wherein the medicament is formulated for the fetal gastrointestinal tract.

17. The method of claim 16, wherein the medicament is formulated for intramembranous fetal transfer.

18. (canceled)

19. The method of claim 3, comprising formulating the medicament to be embedded in microcapsules for extended release characteristics.

20. (canceled)

21. The method of claim 7, comprising surgically emplacing the transmembrane patch at the amniotic membrane.

22. (canceled)

23. (canceled)

24. (canceled)

25. The method of claim 7, comprising placing a sensor in proximity to the fetus to detect one or more physiological conditions of the fetus.

26. The method of claim 25, wherein the sensor is incorporated with the transmembrane patch.

27. The method of claim 25, comprising initiating a signal to detect with the sensor an analyte in an amniotic fluid sample.

28. The method of claim 25, comprising initiating a signal to detect the one or more physiological conditions of the fetus by vibrational sensing.

29. The method of claim 25, comprising initiating a signal to detect the one or more physiological conditions of the fetus by electrical sensing.

30. The method of claim 25, comprising initiating a signal to detect the one or more physiological conditions of the fetus by electromagnetic sensing.

31. The method of claim 25, comprising initiating a signal to control administration of the medicament regulated by a controller in response to the one or more sensed physiological condition.

32. The method of claim 7, comprising administering the medicament by one or more of microneedle injection, microjet, microcapsules, iontophoresis, and sonophoresis

33. The method of claim 7, wherein the transmembrane patch is configured to deliver the medicament in response to a delivery schedule.

34. The method of claim 7, wherein the transmembrane patch is configured to deliver the medicament in response to an external command.

35. (canceled)

36. (canceled)

37. (canceled)

38. The method of claim 3, comprising initiating a signal to a controller to inject the microcapsules through an amniotic membrane by a needleless injector utilizing at least one of a microjet, sonophoresis, or iontophoresis.

39. (canceled)

40. The method of claim 3, comprising initiating a signal to a controller to inject the microcapsules through the amniotic membrane by a needle-based injector non-invasively positioned to the amniotic fluid.

41. The method of claim 40, comprising initiating a signal to a controller to inject the microcapsules transdermally through the maternal skin and through the amniotic membrane by the needle-based injector.

42. (canceled)

43. The method of claim 3, comprising initiating a signal to a controller to transdermally inject the microcapsules through the amniotic membrane to the amniotic fluid by a needle-based injector.

44. (canceled)

45. A method for detecting one or more physiological conditions of a fetus comprising:
placing a device including a transmembrane sensor in contact with an outer wall of an amniotic membrane of the fetus; and
initiating a signal to the transmembrane sensor and a controller of the device to detect the one or more physiological conditions of the fetus.

46. The method of claim 45, comprising initiating a signal to the sensor and the controller to detect the one or more physiological conditions by removing an analyte through the amniotic membrane.

47. The method of claim 46, comprising initiating a signal to the sensor and the controller to remove the analyte through the amniotic membrane by microneedle, sonophoresis, or iontophoresis.

48. The method of claim 45, comprising initiating a signal to the sensor and the controller to detect the one or more physiological conditions by sensing vibrations caused by the fetus or surrounding amniotic tissue.

49. The method of claim 45, comprising initiating a signal to the sensor and the controller to detect the one or more physiological conditions by sensing electrical signals from the fetus or surrounding amniotic tissue.

50. The method of claim 45, comprising initiating a signal to the sensor and the controller to detect the one or more physiological conditions by sensing electromagnetic signals from the fetus or surrounding amniotic tissue.

51. The method of claim 45, comprising initiating a signal to the sensor and the controller to detect one or more of pH, temperature, analyte identity, or analyte concentration.

52. The method of claim 45, comprising initiating a signal to the sensor and the controller to wirelessly report the sensed physiological condition to a remote computing device.

53. The method of claim 45, comprising initiating a signal to the sensor and the controller to surgically reseal tissue over the transmembrane sensor in contact with the outer wall of the amniotic membrane.

54. The method of claim 45, comprising initiating a signal to the sensor and the controller to report the sensed physiological condition to a computing device on a predetermined schedule.

55. The method of claim 45, comprising initiating a signal to the sensor and the controller to report the sensed physiological condition to a computing device in response to one or more queries.

56. The method of claim 45, comprising initiating a signal to the sensor and the controller to report the sensed physiological condition to a computing device based on previous measurements of the physiological condition.

57. The method of claim 45, comprising initiating a signal to the controller to administer a medicament across the amniotic membrane from an injector non-invasively positioned to the amniotic fluid in response to measurements of one or more sensed physiological conditions.

58. A method for delivering a medicament to a fetus comprising:
placing a device including a transmembrane sensor in contact with an outer wall of an amniotic membrane of the fetus;
initiating a signal to the transmembrane sensor and a controller of the device to detect one or more physiological conditions of the fetus; and
initiating a signal the device and the controller to administer the medicament across an amniotic membrane to amniotic fluid of the fetus from an injector non-invasively positioned to the amniotic fluid responsive to one or more sensed physiological conditions.
